(19) United States
(12) Patent Application Publication
(10) Pub. No.: US 2025/0032005 A1
(43) Pub. Date: Jan. 30, 2025

(54) SYSTEM AND METHOD FOR PREDICTIVE ARTIFICIAL INTELLIGENCE (AI) WARNINGS OF LIKELY FOOD-INDUCED GLUCOSE SPIKES

(71) Applicant: Andre Forde, Windermere, FL (US)

(72) Inventor: Andre Forde, Windermere, FL (US)

(21) Appl. No.: 18/784,907

(22) Filed: Jul. 25, 2024

Related U.S. Application Data
(60) Provisional application No. 63/515,426, filed on Jul. 25, 2023.

(51) Int. Cl. A61B 5/145; A61B 5/00; G16H 20/00

(52) U.S. Cl. CPC A61B 5/14532; A61B 5/6803; A61B 5/746; G16H 20/00

(57) ABSTRACT

Disclosed are a system, device, and method for predictive Artificial Intelligence (AI) warnings of likely food-induced glucose spikes, according to one embodiment. An artificial intelligence powered health system includes a continuous glucose monitoring device to capture a real-time glucose level data associated with a body of a wearer. An artificial intelligence server provides a recommendation to improve a health and a well-being of the wearer. A mobile device to generate an alert when an input of a food item consumed by the wearer and/or a behavioral activity performed by the wearer exhibits a health parameter which is greater than a predefined threshold based on a real time health information captured by the continuous glucose monitoring device. A mobile application to recommend an action to the wearer to reduce the health parameter to a normal range, according to this aspect.

### CLAIM OF PRIORITY

This application is a U.S. Utility Conversion Patent Application of U.S. Provisional Patent Application No. 63/515,426 titled ‘ARTIFICIAL INTELLIGENCE POWERED HEALTH COACH CHATBOT’ filed on Jul. 25, 2023. The content of the aforementioned application is incorporated by reference in entirety thereof.

### FIELD OF TECHNOLOGY

This disclosure relates generally to an Artificial Intelligence (AI) powered health coach chatbot and, more particularly, to a system, device, and method for predictive Artificial Intelligence (AI) warnings of likely food-induced glucose spikes.

### BACKGROUND

Eating mindlessly for people with pre-diabetes and diabetes can lead to death by causing repeated blood sugar spikes, significantly contributing to insulin resistance and formation of atherosclerotic plaques. This dramatically increases the risk of heart attacks, strokes, and/or peripheral artery disease, all of which are major causes of death among individuals with diabetes.

Kidney damage (e.g., nephropathy) is another harmful outcome of eating mindlessly which can damage blood vessels in the kidneys, impairing their filtering ability. Over time, this can lead to chronic kidney disease or end-stage renal disease, necessitating dialysis and/or a kidney transplant. Nerve damage (e.g., neuropathy) is also a major concern. Mindless eating elevates blood sugar levels, damaging the nerves, especially in the extremities. This can lead to a loss of sensation, causing unnoticed injuries that can develop into severe infections and potentially necessitate amputations. Severe infections can be life-threatening if not managed properly.

The eyes are also at risk. Mindless eating damages the blood vessels in the retina, leading to diabetic retinopathy. This condition can cause vision problems, blindness, and an increased risk of accidents due to impaired vision, which can indirectly contribute to mortality. Eating mindlessly weakens the immune response, increasing the risk of severe skin infections, urinary tract infections, and respiratory infections. These infections can become life-threatening, especially in individuals with already compromised health. Mindless eating impedes the body's ability to heal wounds. Slow or non-healing wounds, particularly on the feet, can lead to severe infections and gangrene, necessitating amputations. Complications from such infections can be fatal. Insulin resistance and mindless eating negatively impact brain function, leading to cognitive decline, memory problems, and an increased risk of Alzheimer's disease. This decline can reduce a person's ability to manage their health, increasing their mortality risk.

Liver damage, specifically non-alcoholic fatty liver disease (NAFLD), is another significant issue. Eating mindlessly converts excess glucose into fat and stores it in the liver, which can progress to liver inflammation, fibrosis, and cirrhosis. Advanced liver disease can lead to liver failure, a life-threatening condition. Obesity is also linked to insulin resistance. Mindless eating promotes fat storage, exacerbating insulin resistance and increasing the risk of cardiovascular disease, type 2 diabetes, and various cancers, all of which can lead to premature death. The psychological impact of managing these chronic health issues cannot be overlooked. The stress of constant blood sugar management and the complications that arise from poor control can lead to increased rates of depression, anxiety, and stress. This psychological burden can lead to neglect of self-care, further increasing the risk of poor health outcomes and mortality.

### SUMMARY

Disclosed are a system, device, and method for predictive Artificial Intelligence (AI) warnings of likely food-induced glucose spikes.

In one aspect, an artificial intelligence powered health system includes a continuous glucose monitoring device to capture a real-time glucose level data associated with a body of a wearer. An artificial intelligence server provides a recommendation to improve a health and a well-being of the wearer. A mobile device is communicatively coupled with the continuous glucose monitoring device and the artificial intelligence server to generate an alert when an input of a food item consumed by the wearer and/or a behavioral activity (e.g., exercise, drinking, smoking, sleeping, etc.) performed by the wearer exhibits a health parameter which is greater than a predefined threshold based on a real time health information captured by the continuous glucose monitoring device. A mobile application on the mobile device to recommend an action to the wearer to reduce the health parameter to a normal range, according to this aspect.

The artificial intelligence powered health system may include a smart glasses and/or a smart watch communicatively coupled with the mobile application to provide augmented reality based recommendations to the wearer when the alert is generated. A community assistance server (communicatively coupled with the artificial intelligence server) may permit a medical professional (e.g., doctor, a friend, a coach, a peer) to assist the wearer through report generations summarizing the alerts generated by the artificial intelligence model. The community assistance server may provide bidirectional communication between the medical professional and the wearer through the mobile application.

The action may be provided in an empathetic tone that aligns with a current activity of the wearer. The health parameter may be shared with an artificial intelligence training model in an anonymized form to ensure compliance with data protection regulations. The alert may assist the wearer in making healthier options to regulate the health parameter in the normal range and/or to develop habits to reduce a risk of a further deterioration of a health condition of the wearer. The alert may assist the wearer to reduce the uncertainty and stress associated with glucose management.

In another aspect, a glucose monitoring system includes a continuous glucose monitoring device to capture a real-time glucose level data associated with a body of a wearer, a mobile device communicatively coupled with the continuous glucose monitoring device to generate an alert when the continuous glucose monitoring device to detect a spike in a glucose level on the body of the wearer when the wearer is consuming a food, a mobile application on the mobile device to automatically share a contextual data comprising at least one of a location data, a weight data, a visual data, an audio data, a transcript, a voice-response data, and an olfactory data that aligns contemporaneously with a timestamp of the alert and a glucose spike level associated with the alert with an artificial intelligence training model, and an artificial intelligence server to host the artificial intelligence training model to aggregate the contextual data with other contextual data of the wearer and that of other wearers to form a basis for early predictive warnings of imminent glucose spikes based on a continual tagging and training of a predictive artificial intelligence model formed from tagged versions of the contextual data with other contextual data of the wearer and that of other wearers.

In yet another aspect, a method of artificial intelligence powered health system includes capturing a real-time glucose level data associated with a body of a wearer though a continuous glucose monitoring device, providing a recommendation to improve a health and a well-being of the wearer through an artificial intelligence server, determining that a health parameter is greater than a predefined threshold based on a real time health information captured by the continuous glucose monitoring device through a mobile device, generating an alert when an input of at least one food item consumed by the wearer and a behavioral activity performed by the wearer is greater than a predefined threshold based on a real time health information captured by the continuous glucose monitoring device, and commending an action to the wearer to reduce the health parameter to a normal range through a mobile application on the mobile device.

An augmented reality based recommendation may be provided to the wearer when the alert is generated through at least one a smartwatch and a smart glasses communicatively coupled with the mobile application. A medical professional may be permitted to assist the wearer through report generations summarizing the alerts generated by the artificial intelligence model, Bidirectional communication between the medical professional and the wearer through the mobile application may be provided. The action may be suggested in an empathetic tone that aligns with a current activity of the wearer. The health parameter may be shared with the artificial intelligence training model in an anonymized form to ensure compliance with data protection regulations.

The wearer may be assisted in making healthier options to regulate the health parameter in the normal range and to develop habits to reduce a risk of a further deterioration of a health condition of the wearer. The wearer may be assisted to reduce the uncertainty and stress associated with glucose management.

These and other features, aspects, and advantages of the present invention will become better understood with reference to the following description.

The methods and systems disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a network view of an artificial intelligence-powered health system illustrating an artificial intelligence server to provide a recommendation to a wearer of a continuous glucose monitoring device based on real-time health information captured by the continuous glucose monitoring device, according to one embodiment.

FIG. 2 is a user interface view of the artificial intelligence-powered health system of FIG. 1 illustrating a user setting up an alert using his mobile device, according to one embodiment.

FIG. 3 is another user interface view of the artificial intelligence-powered health system of FIG. 1 illustrating the automatic presentation of a menu of activities triggered by glucose levels recorded by a continuous glucose monitoring device of the user, according to one embodiment.

FIG. 4 is another user interface view of the artificial intelligence-powered health system of FIG. 1 illustrating the user submitting the details of activity type from the menu of activities, according to one embodiment.

FIG. 5 is another user interface view of the artificial intelligence-powered health system of FIG. 1 illustrating the photographs of food items being uploaded on the system that are consumed by the user, according to one embodiment.

FIG. 6 is a user interface view of a physician portal of the artificial intelligence-powered health system of FIG. 1, according to one embodiment.

FIG. 7 is another user interface view of the physician portal of the artificial intelligence-powered health system of FIG. 1 illustrating various tabs for the physician to navigate the patients' data, according to one embodiment.

FIG. 8 is a user interface view of the physician portal of the artificial intelligence-powered health system of FIG. 1 illustrating the features offered by the system to sort patient data, according to one embodiment.

FIG. 9 is a table view illustrating the analytical data stored in the database of the artificial intelligence-powered health system of FIG. 1, according to one embodiment.

FIG. 10 is a process flow of the artificial intelligence-powered health system of FIG. 1 illustrating the steps involved in generating a recommendation to a wearer of a continuous glucose monitoring device, according to one embodiment.

FIG. 11 is another process flow of the artificial intelligence-powered health system of FIG. 1 illustrating the steps involved in assisting the wearer to reduce the uncertainty and stress associated with glucose management, according to one embodiment.

Other features of the present embodiments will be apparent from the accompanying drawings and from the detailed description that follows.

### DETAILED DESCRIPTION

Disclosed are a system, device, and method for predictive Artificial Intelligence (AI) warnings of likely food-induced glucose spikes (e.g. Dula™), according to one embodiment. This innovative system comprises two main components: a continuous glucose monitor (CGM) and a mobile application, according to one embodiment.

In one embodiment, an artificial intelligence-powered health system 130 includes a continuous glucose monitoring device 112 to capture a real-time glucose level data associated with a body of a wearer 114. An artificial intelligence server 100 provides a recommendation 120 to improve a health and a well-being of the wearer 114. A mobile device 110 is communicatively coupled with the continuous glucose monitoring device 112 and the artificial intelligence server 100 to generate an alert 202 when an input of a food item consumed by the wearer 114 and/or a behavioral activity (e.g., exercise, drinking, smoking, sleeping, etc.) performed by the wearer 114 exhibits a health parameter which is greater than a predefined threshold based on a real-time health information 118 captured by the continuous glucose monitoring device 112. A mobile application 116 on the mobile device 110 recommends an action to the wearer 114 to reduce the health parameter to a normal range, according to this aspect.

The artificial intelligence-powered health system 130 may include a smart glasses 107 and/or a smartwatch 109 communicatively coupled with the mobile application 116 to provide augmented reality-based recommendations 120 to the wearer 114 when the alert 202 is generated. A community assistance server 122 (communicatively coupled with the artificial intelligence server 100) may permit a medical professional 124 (e.g., a doctor, a friend, a coach, a peer) to assist the wearer 114 through report generations summarizing the alerts 202 generated by the artificial intelligence model 126. The community assistance server 122 may provide bidirectional communication between the medical professional 124 and the wearer 114 through the mobile application 116.

The action may be provided in an empathetic tone that aligns with a current activity of the wearer 114. The health parameter may be shared with an artificial intelligence training model in an anonymized form to ensure compliance with data protection regulations. The alert 202 may assist the wearer 114 in making healthier options to regulate the health parameter in the normal range and/or to develop habits to reduce a risk of a further deterioration of a health condition of the wearer 114. The alert 202 may assist the wearer 114 to reduce the uncertainty and stress associated with glucose management.

In another embodiment, a glucose monitoring system includes a continuous glucose monitoring device 112 to capture a real-time glucose level data associated with a body of a wearer 114, a mobile device 110 communicatively coupled with the continuous glucose monitoring device 112 to generate an alert 202 when the continuous glucose monitoring device 112 to detect a spike in a glucose level on the body of the wearer 114 when the wearer 114 is consuming a food, a mobile application 116 on the mobile device 110 to automatically share a contextual data comprising a location data, a weight data, a visual data, an audio data, a transcript, a voice-response data, and an olfactory data that aligns contemporaneously with a timestamp of the alert 202 and a glucose spike level associated with the alert 202 with an artificial intelligence training model, and an artificial intelligence server 100 to host the artificial intelligence training model (e.g., AI model 126) to aggregate the contextual data with other contextual data of the wearer 114 and that of other wearers 114 to form a basis for early predictive warnings of imminent glucose spikes based on a continual tagging and training of a predictive artificial intelligence model 126 formed from tagged versions of the contextual data with other contextual data of the wearer 114 and that of other wearers.

In yet another embodiment, a method of artificial intelligence-powered health system 130 includes capturing a real-time glucose level data associated with a body of a wearer 114 though a continuous glucose monitoring device 112, providing a recommendation 120 to improve a health and a well-being of the wearer 114 through an artificial intelligence server 100, determining that a health parameter is greater than a predefined threshold based on a real time health information 118 captured by the continuous glucose monitoring device 112 through a mobile device 110, generating an alert 202 when an input of at least one food item consumed by the wearer 114 and a behavioral activity performed by the wearer 114 is greater than a predefined threshold based on a real-time health information 118 captured by the continuous glucose monitoring device 112, and commending an action to the wearer 114 to reduce the health parameter to a normal range through a mobile application 116 on the mobile device 110.

An augmented reality based recommendations 120 may be provided to the wearer 114 when the alert 202 is generated through at least one a smartwatch 109 and a smart glasses 107 communicatively coupled with the mobile application 116. A medical professional 124 may be permitted to assist the wearer 114 through report generations summarizing the alerts 202 generated by the artificial intelligence model 126, Bidirectional communication between the medical professional 124 and the wearer 114 through the mobile application 116 may be provided. The action may be suggested in an empathetic tone that aligns with a current activity of the wearer 114. The health parameter may be shared with the artificial intelligence training model in an anonymized form to ensure compliance with data protection regulations.

The wearer 114 may be assisted in making healthier options to regulate the health parameter in the normal range and to develop habits to reduce a risk of a further deterioration of a health condition of the wearer 114. The wearer 114 may be assisted to reduce the uncertainty and stress associated with glucose management.

### Mental Awareness Application Generates Training Data for Dula™ AI

The application used in the training phase offers significant mental and psychological benefits, making it a novel and important tool even without the AI component, according to one embodiment. This mobile application 116 is designed to integrate continuous glucose monitoring with a dietary log and human input, all triggered in a timely manner based on physiological responses, according to one embodiment. This unique combination addresses the psychological and behavioral aspects of managing glucose levels, providing users with a comprehensive and supportive tool, according to one embodiment.

One of the key benefits of the application is its ability to promote mindful eating, according to one embodiment. By integrating continuous glucose monitoring with a dietary log, the application encourages users 200 to be more aware of their eating habits and the impact of different foods on their glucose levels, according to one embodiment. When a glucose spike is detected, the application prompts users to log what they have eaten (e.g., food type 312 and its estimated portion size 314 of user interface view 350), fostering a habit of mindful eating, according to one embodiment. This practice helps users 200 become more conscious of their food choices and encourages healthier eating patterns, which can lead to better overall health and well-being, according to one embodiment.

The application provides real-time feedback to users 200, which is crucial for immediate behavioral adjustments, according to one embodiment. When a glucose spike occurs, users receive timely prompts to log their food intake and activities, according to one embodiment. This immediate feedback loop creates a sense of accountability, as users 200 can see the direct consequences of their dietary choices on their glucose levels, according to one embodiment. This real-time interaction helps users 200 make more informed decisions about their diet and lifestyle, reinforcing positive behaviors and discouraging negative ones, according to one embodiment.

Managing glucose levels can be a source of anxiety for many individuals, particularly those with diabetes or prediabetes, according to one embodiment. The application helps reduce this anxiety by providing users 200 with continuous monitoring and timely alerts, ensuring they are always aware of their glucose levels, according to one embodiment. This constant vigilance allows users 200 to feel more in control of their health, reducing the uncertainty and stress associated with glucose management, according to one embodiment. By offering a structured and supportive approach, the application empowers users 200 to take proactive steps in managing their condition, according to one embodiment.

The application's requirement for users 200 to log their dietary intake and activities encourages self-reflection, according to one embodiment. By regularly documenting their actions and observing the corresponding physiological responses, users can identify patterns and triggers that lead to glucose spikes, according to one embodiment. This self-awareness is critical for behavior modification, as it allows users 200 to pinpoint specific habits that need to be changed, according to one embodiment. The application supports this process by providing a detailed record of dietary choices and glucose responses, facilitating informed and sustained behavior change, according to one embodiment.

The novelty of this application lies in its simultaneous use of sensors for continuous glucose monitoring combined with a dietary log and human input triggered in a timely manner based on physiological responses, according to one embodiment. Unlike traditional methods that rely solely on periodic glucose checks or manual food logs, this application integrates real-time physiological data with user input, creating a dynamic and interactive tool for glucose management, according to one embodiment. By combining continuous glucose monitoring with real-time dietary logging and user feedback, the application bridges the gap between technology and personal accountability, according to one embodiment. This integration ensures that users receive immediate and relevant feedback, enhancing the effectiveness of their glucose management efforts, according to one embodiment.

The application addresses both the physiological and psychological aspects of glucose management, according to one embodiment. It not only monitors glucose levels but also encourages healthy eating habits, reduces anxiety, and promotes self-reflection and behavior change, according to one embodiment. While the application is valuable on its own, it also serves as a foundation for future AI enhancements, according to one embodiment. The data collected during the training phase can be used to develop sophisticated AI models that provide even more personalized and proactive health recommendations, according to one embodiment.

The application used in the training phase offers significant mental and psychological benefits, promoting mindful eating, providing real-time feedback, reducing anxiety, and encouraging behavior modification, according to one embodiment. Moreover, the training data collection phase is crucial for developing a robust AI-enhanced glucose monitoring and behavioral guidance system, according to one embodiment. This phase involves gathering comprehensive and contextual data from both historical sources and initial users who primarily use the mobile app to practice mindful eating before the neural network is fully formed and the AI has enough data to train a model and perform inference, according to one embodiment. The system begins by collecting extensive historical data from existing sources, including medical records, dietary logs, and/or glucose monitoring studies, according to one embodiment. This data, which is anonymized to ensure compliance with data protection regulations, is integrated into the system's database using data import and integration tools, according to one embodiment. This historical data provides a foundational dataset that helps initialize the AI model 126, according to one embodiment.

Next, the system focuses on collecting data from a large number of initial users, according to one embodiment. These users are equipped with continuous glucose monitors (CGMs) and the associated mobile application, according to one embodiment. The CGM devices 112 are attached to the user's body, typically on the upper arm or abdomen, and configured according to the manufacturer's instructions, according to one embodiment. Users install the mobile application on their smartphones and establish a secure Bluetooth connection with the CGM device 112, according to one embodiment.

During this phase, users primarily use the mobile app to practice mindful eating, according to one embodiment. They are prompted to log their food intake and activities, providing detailed contextual information, according to one embodiment. This user interaction is crucial for creating a rich dataset that captures the relationship between dietary choices and glucose levels, according to one embodiment. The CGM devices 112 continuously monitor the users' glucose levels and transmit this data to the mobile application at regular intervals, according to one embodiment. The mobile app analyzes the incoming glucose data in real time and detects spikes in glucose levels, according to one embodiment. When a spike is detected, the app alerts the user and prompts them to provide contextual information about their recent activities and dietary choices, according to one embodiment.

Users 200 can input contextual information through various methods, including verbal input, photos, and text, according to one embodiment. The app uses voice recognition technology to transcribe spoken descriptions, allows users 200 to take photos 502 of their meals or environment, and offers text input fields for manual descriptions, according to one embodiment. This contextual information is integrated with the glucose level data, associating each spike with the user-provided details, according to one embodiment. The mobile application 116 also leverages the smartphone's GPS module to track the user's location in real time, according to one embodiment. When a glucose spike is detected, the app logs the GPS location where the spike occurred, creating a comprehensive dataset that includes time, location, weight, and/or user activities, according to one embodiment. This location data is continuously logged and associated with the timestamp of each glucose reading, according to one embodiment.

All collected data, including glucose levels, user input, GPS logs, and weight, is securely stored in a cloud-based database (e.g., using the community assistance server 122), according to one embodiment. The data is anonymized to protect user privacy and ensure compliance with relevant data protection regulations, according to one embodiment. Robust encryption protocols are implemented for data transmission between the CGM device and the mobile application, as well as for data storage in the cloud, according to one embodiment. To ensure the system's effectiveness and scalability, several technical considerations are addressed. For example, robust encryption protocols to protect data transmission and storage, according to one embodiment. All data is anonymized, and users are informed about data collection practices, according to one embodiment. Necessary consents are obtained for using their data for AI model 126 training, according to one embodiment. The mobile application 116 is designed to be compatible with various CGM devices and smartphone operating systems (iOS and Android), according to one embodiment. The system is designed to handle data from a large number of users, ensuring it can scale efficiently as the user base grows, according to one embodiment.

In summary, the training data collection phase integrates continuous glucose monitoring with user-provided contextual information, weight, and GPS data logging, according to one embodiment. By collecting and analyzing data from both historical sources and a large number of initial users, the system ensures that the AI model 126 receives high-quality, detailed data necessary for accurate predictions and effective behavioral guidance, according to one embodiment. This comprehensive approach enables the development of a robust and effective system for managing glucose levels and providing personalized health recommendations, according to one embodiment.

### Tagging of Training Data for Dula™ AI

The tagging phase is the next step in preparing the collected data for training the Dula™ AI model 126, according to one embodiment. This phase involves annotating and organizing the training data with the help of trained medical professionals 124, such as doctors, nutritionists, physician assistants, and other experts, according to one embodiment. These professionals meticulously examine the training data to identify specific trigger points for generating alerts 202 and categorize these alerts 202 to enhance the training data with actionable recommendations 120, according to one embodiment.

The process begins by hiring a team of medical professionals 124 with expertise in understanding the physiological and behavioral aspects of glucose management, according to one embodiment. These experts undergo thorough training to familiarize themselves with the system's objectives, the data types they will be working with, and the importance of accurate data annotation, according to one embodiment. Given access to thousands of instances of training data, each instance consists of a detailed record of a glucose spike, including contextual information provided by users, according to one embodiment. They meticulously review each data point, analyzing contextual information such as the type of food consumed, the time of day, the location, and other relevant details, according to one embodiment.

The experts identify specific trigger points when alerts 202 should be generated based on observed patterns, such as significant glucose spikes following the consumption of particular foods or engaging in certain activities. Each identified trigger point is tagged with comprehensive information, including the type of food 312, time of day, location, and/or the user's activities leading up to the spike, according to one embodiment. The professionals then categorize the alerts 202 to enhance the training data by providing detailed descriptions of the recommended actions to be taken when an alert 202 is triggered, according to one embodiment. This categorization includes specific dietary recommendations 120, activity modifications, and other behavioral adjustments designed to help users manage their glucose levels more effectively, according to one embodiment.

The system integrates various types of input data, including verbal descriptions, photos 502, and text entries (e.g., adding notes using 506 tab of the user interface view 550), with the glucose level data, GPS logs, and weight measurements, creating a rich, multifaceted dataset that captures a comprehensive view of each user's glucose management context, according to one embodiment. Medical professionals 124 conduct a comparative analysis of the GPS locations, times of day, and descriptions of why spikes occurred with the specific foods consumed at those times, according to one embodiment. They generate tags that reflect these comparisons, ensuring that the dataset captures the nuanced relationships between user behavior and glucose levels, according to one embodiment.

The collected data is cleaned to remove any inconsistencies or errors, which involves standardizing formats, correcting inaccuracies, and ensuring all data entries are complete and accurate, according to one embodiment. Data cleaning is crucial for maintaining the quality and reliability of the dataset, according to one embodiment. Once cleaned, the data undergoes preprocessing to prepare it for model training, according to one embodiment. This involves normalizing data values, handling missing data points, and transforming the data into a format suitable for machine learning algorithms, according to one embodiment. Preprocessing ensures that the data fed into the AI model 126 is consistent and accurate, enhancing the model's ability to learn and make precise predictions, according to one embodiment.

In summary, the tagging phase relies on trained medical professionals 124 to annotate and organize the collected data, identifying trigger points for alerts 202 and categorizing them with actionable recommendations 120, according to one embodiment. By integrating various data types and conducting a comparative analysis of contextual information, these experts enrich the dataset, according to one embodiment. The cleaned and preprocessed data is then ready for model training, ensuring the AI model 126 can accurately predict and manage glucose spikes, ultimately providing users with effective behavioral guidance and personalized health recommendations 120, all while maintaining data anonymity to protect user privacy, according to one embodiment.

### Model Generation Phase for Dula™ AI

The model generation phase is a pivotal step in developing the Dula™ AI-enhanced glucose monitoring and behavioral guidance system, according to one embodiment. This phase involves transforming the annotated and preprocessed data into a sophisticated machine learning model capable of making accurate predictions and providing actionable recommendations 120, according to one embodiment. It begins with data preparation, where the cleaned and annotated dataset is divided into training, validation, and testing subsets, according to one embodiment. Feature engineering follows, extracting key features such as glucose levels, dietary information, time of day, location data, and user activities, according to one embodiment. Additional derived features like the rate of glucose level change and the frequency of specific food consumption are also generated to provide the model with comprehensive inputs, according to one embodiment.

Next, the model selection and training process takes place, according to one embodiment. Various machine learning algorithms are evaluated to determine the most suitable model, considering factors such as the ability to handle complex relationships, interpretability, and computational efficiency, according to one embodiment. Algorithms like decision trees, random forests, gradient boosting machines, and neural networks are commonly considered, according to one embodiment. Hyperparameter tuning is performed to optimize the model's performance, using techniques like grid search and random search to identify optimal values, according to one embodiment. The training dataset and tagging data is then used to train the model, employing advanced techniques such as cross-validation to ensure the model generalizes well to unseen data, according to one embodiment.

Validation testing follows, where the trained Dula™ AI model 126 is evaluated on the validation dataset to assess its performance, according to one embodiment. Metrics such as accuracy, precision, recall, F1 score, and AUC-ROC quantify the model's effectiveness, according to one embodiment. Based on these results, the model may undergo further refinement, adjusting hyperparameters, retraining with different subsets, or selecting a different algorithm if necessary, according to one embodiment. This iterative process continues until the model achieves satisfactory performance, according to one embodiment.

The refined model is then subjected to final testing using the testing dataset, providing an unbiased evaluation of its performance and ensuring readiness for deployment, according to one embodiment. Performance metrics are recalculated to verify the model's accuracy and reliability, according to one embodiment. Comparative analysis against baseline models and previous iterations ensures significant improvement, with additional robustness checks to confirm general applicability across different demographic groups and conditions, according to one embodiment.

Finally, the model deployment phase involves integrating the model into the existing glucose monitoring system, according to one embodiment. Deployment frameworks like Docker and Kubernetes streamline this process, ensuring scalability and efficient real-time data processing from the continuous glucose monitor, according to one embodiment. Continuous monitoring tools track the model's performance in real-world conditions, monitoring prediction accuracy, user feedback, and system stability, according to one embodiment. Regular maintenance, including updating the model with new data and periodic retraining, ensures sustained accuracy and effectiveness over time, according to one embodiment.

In summary, the Dula™ AI model 126 generation phase meticulously prepares, trains, validates, and tests the machine learning model, according to one embodiment. Through comprehensive data preparation, feature engineering, algorithm selection, hyperparameter tuning, and rigorous validation and testing, a robust AI model 126 is developed, according to one embodiment. This model is then seamlessly integrated into the glucose monitoring system, providing personalized and actionable health recommendations 120 to enhance users' ability to manage their glucose levels effectively, according to one embodiment.

Detailed Technical Description: Inference Phase with the Dula™ AI Model 126

The inference phase is the stage where the Dula™ AI Model 126 is deployed to provide real-time insights and recommendations to users 200, according to one embodiment. This phase leverages the power of the trained model to deliver actionable health guidance, ultimately helping users manage their glucose levels more effectively and improve their overall health, according to one embodiment. The process begins with real-time monitoring, where the Continuous Glucose Monitor (CGM) device 112 continuously tracks the user's glucose levels and transmits this data to the mobile application 116 via Bluetooth or other wireless communication methods, according to one embodiment. This real-time data stream is then relayed to the cloud-based Dula™ AI Model 126 for processing and analysis, where the mobile app integrates real-time glucose data with other contextual information such as recent dietary inputs, physical activity, and GPS location, providing a comprehensive view of the user's current physiological state, according to one embodiment.

When the Dula™ AI Model 126 predicts a potential glucose spike based on this integrated data set and patterns learned during the training phase, the mobile application 116 generates proactive alerts 202 to notify the user 200 before the spike occurs, according to one embodiment. These alerts 202 are delivered through various channels, including push notifications on the smartphone, SMS, or emails, ensuring the user 200 receives timely warnings regardless of their current activity or location, according to one embodiment. The app also provides personalized recommendations 120 to help the user prevent glucose spikes, offering tailored suggestions based on the user's historical data, preferences, and current context, according to one embodiment. Examples include suggesting alternative food choices, recommending a brief walk or exercise, or advising hydration, according to one embodiment. The app engages the user 200 interactively, asking questions about their recent activities and offering advice based on their responses, helping users 200 understand the impact of their choices and encouraging positive behavior changes, according to one embodiment.

As users interact with the app and follow the recommendations 120, new data is continuously collected, including the user's responses to alerts, adherence to recommendations 120, and subsequent glucose level readings, according to one embodiment. This feedback is sent back to the cloud-based Dula™ AI Model 126, creating a continuous learning loop. The Dula™ AI Model 126 uses this feedback to refine its algorithms and improve its accuracy over time, with machine learning techniques such as reinforcement learning applied to enhance the model's predictive capabilities, according to one embodiment. Regular updates and retraining of the model ensure it remains effective and adapts to changes in user behavior and physiology, according to one embodiment.

Creative approaches and embodiments during inference can further enhance the system's effectiveness, according to one embodiment. Integration with other wearable devices 108, such as smartwatches 109 and fitness trackers, provides additional data points like heart rate, physical activity levels, and sleep patterns, enabling more accurate predictions and personalized recommendations 120, according to one embodiment. Incorporating voice-activated assistants like Amazon Alexa or Google Assistant allows users 200 to receive alerts 202 and recommendations 120 through voice commands, enhancing accessibility and convenience, according to one embodiment. Creating a community platform within the app where users 200 can share experiences, tips, and support fosters a sense of camaraderie and accountability, with group challenges and peer support motivating users to adhere to healthy behaviors, according to one embodiment. Implementing gamification elements such as rewards, achievements, and progress tracking makes the experience more engaging and motivates users to follow recommendations consistently, according to one embodiment. Additionally, augmented reality (AR) features can be used to visually demonstrate the impact of certain foods on glucose levels, with users 200 scanning food items with their smartphone camera to see an AR overlay showing its potential impact, according to one embodiment.

In summary, the inference phase involves deploying the Dula™ AI Model 126 to provide real-time insights and recommendations 120 through continuous glucose monitoring, proactive alerts 202, personalized behavioral recommendations, and continuous learning, according to one embodiment. By integrating creative approaches such as wearable integration, voice-activated assistance, social support, gamification, and AR features, the system enhances the user experience and effectiveness, ultimately aiming to improve users' overall health through proactive, AI-driven insights, according to one embodiment.

### Dula™ Glucose Monitor: Continuous Glucose Monitoring Device 112

The Dula™ Glucose Monitor is a cutting-edge continuous glucose monitoring (CGM) device 112 designed to seamlessly integrate with the Dula™ AI Model 126, offering real-time glucose level data and personalized health recommendations 120, according to one embodiment. This device can be implemented in various ways to accommodate user comfort and lifestyle, ensuring continuous and accurate monitoring of glucose levels, according to one embodiment. One of the primary implementations is a small, adhesive patch worn on the upper arm, according to one embodiment. This patch contains a tiny sensor that measures interstitial glucose levels just below the skin, providing discreet and comfortable long-term wear, according to one embodiment. It is easily accessible for calibration and data upload, allowing minimal interference with daily activities, according to one embodiment. Alternatively, the Dula™ Glucose Monitor can be a subcutaneous sensor inserted just under the skin, typically on the abdomen or upper thigh, according to one embodiment. This sensor continuously measures glucose levels and wirelessly transmits data to the Dula™ mobile app, according to one embodiment. The subcutaneous sensor offers highly accurate readings, can be worn for extended periods (e.g., up to 14 days), and is less visible than external patches, according to one embodiment.

Another innovative implementation integrates the Dula™ Glucose Monitor into existing wearable devices 108, such as smartwatches 109 or fitness trackers, according to one embodiment. This integration places the glucose sensor in the wearable device 108, allowing it to continuously monitor glucose levels alongside other health metrics like heart rate and activity levels, according to one embodiment. This method provides a consolidated view of the user's health data, combining convenience and fashion for users who already wear such devices, according to one embodiment. Additionally, the Dula™ Glucose Monitor can be designed as disposable sensor patches that can be applied to different body parts, such as the lower back or the side of the torso, according to one embodiment. These patches are intended for single use, providing continuous glucose monitoring for a set period (e.g., 7 days) before being replaced, according to one embodiment. This approach offers hygienic and convenient monitoring for users 200 who prefer not to maintain long-term devices and allows flexibility in placement based on user comfort and preference, according to one embodiment.

The Dula™ Glucose Monitor features real-time data transmission to the Dula™ mobile application, ensuring users 200 receive up-to-date information and alerts 202 about their glucose levels, according to one embodiment. Its seamless integration with the Dula™ AI Model 126 enables the AI to analyze glucose data alongside other contextual information, such as dietary intake and physical activity, to provide personalized recommendations and proactive alerts, according to one embodiment. The mobile application 116 is designed with a user-friendly interface that displays glucose trends, historical data, and real-time alerts 202, making it easy for users to log their meals, activities, and other relevant information, according to one embodiment. The device is designed with user comfort in mind, being lightweight, discreet, and easy to wear, allowing users 200 to continue their daily activities without disruption, according to one embodiment.

Equipped with a long-lasting battery, the Dula™ Glucose Monitor ensures uninterrupted monitoring, with options for rechargeable or replaceable batteries for user convenience, according to one embodiment. Data transmitted from the Dula™ Glucose Monitor to the mobile application is encrypted and securely stored, ensuring user privacy and data protection, according to one embodiment. Overall, the Dula™ Glucose Monitor is a versatile and advanced CGM device that provides continuous and accurate glucose monitoring, according to one embodiment. Its seamless integration with the Dula™ AI Model 126 ensures real-time data transmission, personalized recommendations, and proactive alerts, making it an essential tool for effective glucose management and overall health improvement, according to one embodiment.

One Embodiment of an Integrated system:

The CGM 112 is a device that continuously tracks glucose levels in the user's body, according to one embodiment. It connects to the user's mobile phone via Bluetooth or another connection mechanism, according to one embodiment. When the CGM detects a glucose spike, it sends data to the mobile application 116, which then alerts 202 the user 200 through various methods such as calls, vibrations, or notifications on synced devices like an iPhone or iMac, according to one embodiment.

Upon detecting a spike, the application prompts the user 200 to provide contextual information about their activities, particularly their eating habits, according to one embodiment. This information can be supplied verbally, through photos, or via text, according to one embodiment. Additionally, the application can use GPS to log the location of the spike, creating a detailed map and timeline of glucose level fluctuations, according to one embodiment.

A significant aspect of this AI-powered health system 130 may be the integration of artificial intelligence (AI), according to one embodiment. The application collects and analyzes the data provided by the user to understand the causes of glucose spikes, according to one embodiment. Over time, the AI learns from the aggregated, anonymized data of all users 200, identifying patterns and triggers for glucose spikes, according to one embodiment. Based on this learned behavior and location data, the AI can proactively alert users to potential spikes before they occur, according to one embodiment. For instance, it might warn a user when they approach a fast-food restaurant known to trigger spikes, according to one embodiment.

The AI-powered health system 130 may also include a behavioral coaching component, according to one embodiment. The application may act as a virtual coach, engaging users in conversations about their food choices and activities when a spike occurs, according to one embodiment. This may help users 200 mentally reframe their actions to better manage their glucose levels, according to one embodiment. Optionally, a human life coach can be integrated into the system for additional support, according to one embodiment.

The benefits of this invention are multifaceted, according to one embodiment. It improves glucose management by providing real-time alerts and insights into glucose levels, helping users 200 make better dietary and lifestyle choices, according to one embodiment. The AI-driven recommendations reduce the frequency of glucose spikes, which is crucial for managing chronic conditions like diabetes, hyperglycemia, metabolic syndrome, and pancreatic disorders, according to one embodiment. Additionally, the system offers data-driven insights by providing detailed visualizations and analyses of glucose levels over time, aiding users in understanding their personal glucose trends and triggers, according to one embodiment.

In conclusion, this invention combines a continuous glucose monitor with an intelligent mobile application that not only alerts users to glucose spikes but also engages them in identifying and modifying the behaviors that cause these spikes, according to one embodiment. By leveraging AI, the system evolves to provide personalized, preemptive recommendations, ultimately helping users 200 maintain healthier glucose levels and improve their overall well-being, according to one embodiment.

In accordance with one embodiment, a system for providing health recommendations 120 is disclosed. In some embodiments, the system is an Artificial Intelligence (AI) powered health coach chatbot system that may be hosted on an AI server 100. A user 200 may access the system by downloading and installing a system application (“app” e.g., mobile application 116) on a mobile device 110.

In certain embodiments, the system includes a transceiver 102, a memory 104 and an AI-based processor 106. The transceiver 102 is configured to receive real-time health information 118 associated with a user from a wearable device 108 worn by the user. In some embodiments, the real-time health information 118 is associated with real-time glucose level of the user 200. The transceiver 102 is further configured to receive inputs associated with at least one of a food item consumed by the user 200 and an activity (e.g., exercise, drinking, smoking, sleeping, etc.) performed by the user 200. The memory 104 is configured to store the real-time health information and the inputs.

The AI-based processor 106 is communicatively coupled with the memory 104 and the transceiver 102. The AI-based processor 106 is configured to obtain the real-time health information 118 from the memory 104, and determine that a health parameter (e.g., glucose level) associated with the user 200 is greater than a predefined threshold based on the real-time health information 118. Responsive to determining that the health parameter associated with the user 200 is greater than the predefined threshold, the AI-based processor 106 obtains the inputs. The AI-based processor 106 then determines a recommended action to reduce the health parameter (e.g., the glucose level) based on the inputs. The AI-based processor 106 then outputs, via the transceiver 102, the recommendation action to the mobile device 110. The same concept may operate in the converse, where a health parameter is below a predefined threshold, and the recommended action is for increasing it.

The system (e.g., AI-powered health system 130), as described in the present disclosure, provides real-time glucose insights to the user 200 and user's doctor, and assists the user 200 in making better lifestyle decisions throughout the diabetic disease. Further, the system establishes an emotional connection with the user 200 and recommends positive alternatives to user's bad habits and lifestyle decisions. Furthermore, the system assists the user's doctor in gaining deeper insights into user's metabolic health and glucose response, which leads to better hyper-personalized treatment plans and improved patient outcomes.

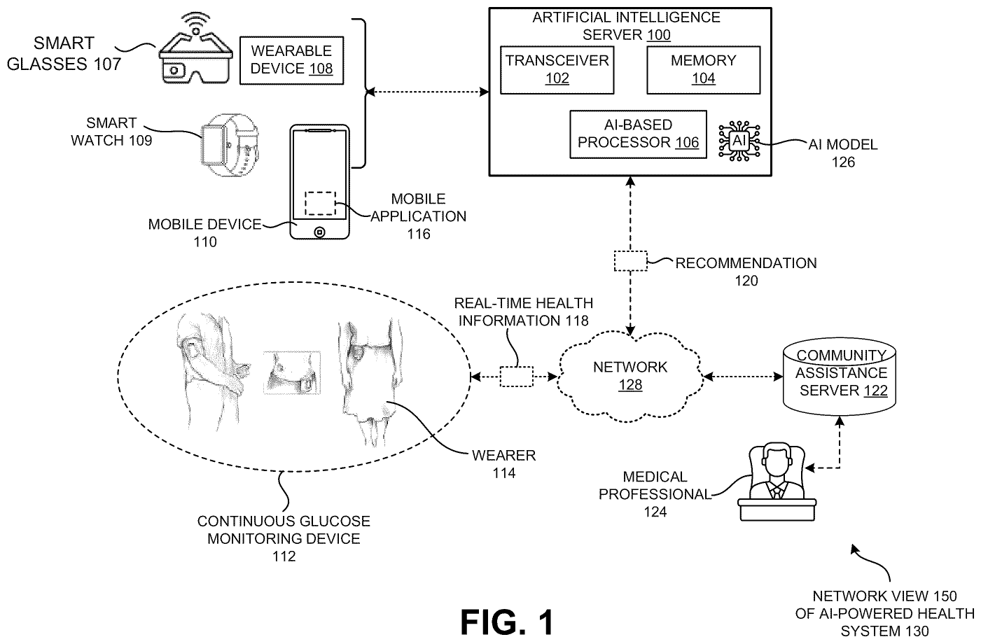

FIG. 1 is a network view 150 of an artificial intelligence-powered health system 130 illustrating an artificial intelligence server 100 to provide a recommendation 120 to a wearer 114 of a continuous glucose monitoring device 112 based on a real-time health information 118 captured by the continuous glucose monitoring device 112, according to one embodiment. FIG. 1 depicts a block diagram of an Artificial Intelligence (AI) powered health system 130 in accordance with one embodiment. The system includes a transceiver 102, a memory 104 and an AI-based processor 106. The system is communicatively connected with a wearable device 108 and a mobile device 110, via a network 128. In certain embodiments, the system is hosted on an AI server 100.

In certain embodiments, the wearable device 108 is a glucose monitoring device worn by a user 200, and is configured to continuously monitor glucose level of the user 200. The mobile device 110 may be a mobile phone, a laptop, a tablet, a computer, or any other communication device associated with the user 200.

In operation, the user 200 downloads and installs an application (“app”) associated with the system on the mobile device 110. Responsive to installing the app, the user 200 registers as a new user by transmitting, via the transceiver 102, user name, health details, and other information associated with the user to the system. Registering on the system enables the system to provide personalize healthcare recommendation 120 and services to the user.

Responsive to registering on the app (or the system), the user pairs the wearable device 108 with the app/system. By pairing the wearable device 108 with the system, the user enables the system to continuously receive user's glucose level data from the wearable device 108. Specifically, responsive to pairing the wearable device 108 with the system, the transceiver 102 begins to receive real-time glucose level data (or “health information” associated with the user) from the wearable device 108. The transceiver 102 sends the received real-time glucose level data to the memory 104 for storage purpose, and the AI-based processor 106 continuously fetches the real-time glucose level data from the memory 104 and analyzes the data.

The user 200 further sets lower threshold and higher threshold of glucose levels on the system, beyond which the AI-based processor 106 outputs an alert notification to the mobile device 110 and recommends remedial actions. For example, the AI-based processor 106 outputs an alert notification (visual and/or audible) to the mobile device 110 when the real-time glucose level of the user exceeds the higher threshold or drops below the lower threshold. The alert notification may provide an indication to the user 200 that the glucose level has reached critical range, and hence the user may exercise caution.

In an exemplary embodiment, when the real-time glucose level of the user 200 exceeds the higher threshold (or drops below the lower threshold), the AI-based processor 106, via the transceiver 102, additionally requests the user 200 to provide details of the food consumed by the user, activities (e.g., smoking, drinking, exercising, sleeping, etc.) performed, and/or the like. Responsive to receiving the request from the AI-based processor 106 on the mobile device 110, the user 200 provides the inputs associated with the food or drinks consumed and/or the activities performed to the transceiver 102, via the mobile device 110. The inputs may be in the form of text or verbal inputs provided by the user 200, or images of the food items consumed. The transceiver 102 sends the received inputs to the memory 104 for storage purpose, and the AI-based processor 106 fetches the inputs from the memory 104.

Responsive to fetching the inputs from the memory 104, the AI-based processor 106 analyzes the inputs to determine one or more recommended actions to reduce the glucose level. For example, based on the inputs, if AI-based processor 106 determines that one or more food items cause an increase in glucose level in the user 200, the AI-based processor 106 recommends the user 200 to stop or minimize intake of such food items. Similarly, if one or more activities (e.g., excessive drinking and/or smoking) cause an increase in the glucose level of the user, the AI-based processor 106 recommends the user 200 to stop or minimize the intensity/duration of the activity.

In this manner, the system assists user 200 in managing glucose levels, and hence living a healthy life. The system provides emotional and coaching support to the user 200 by monitoring user's daily/real-time glucose levels. In certain embodiments, the system additionally provides assistance during mental illness, and provides sports coaching, business coaching, spiritual coaching, teen coaching, and/or the like. Furthermore, the system may be used to monitor and coach users with a variety of chronic diseases including, but not limited to, hypertension, asthma, fatty liver disease, mental illness, Alzheimer's disease, and/or the like, using condition-relevant parameters (e.g., real-time blood pressure for hypertension).

The system enables user 200 to be more aware of certain food items and/or activities that trigger glucose response in the user, and hence the system provides “personalized” support to the user 200. The system further enables the user 200 to customize the app so that the user feels more emotionally connected to the experience provided by the system, based on personal preferences.

In certain embodiments, the system also transmits the real-time glucose level data of the user to a physician or the user's doctor (specifically, doctor's device) so that the physician may monitor the user's health in real-time and determine triggers to the glucose levels. Specifically, the real-time glucose level data empowers the doctor to gain deeper insights into the user's metabolic health and glucose response which leads to better hyper-personalized treatment plans and improved patient outcomes.

In some embodiments, the system (or the AI-based processor 106) utilizes machine learning algorithms to provide personalized, human-like coaching to help users suffering from chronic diseases manage their health effectively. The system reads glucose and metabolic health data from various wearable and continuous monitoring devices, captures daily metabolic levels in real-time, and shares them with the user and user's doctor(s). The system also provides emotional support, nutritional advice, and personalized coaching in multiple languages and multi-ethnic personas. The machine learning algorithms used by the system are designed to learn from the user's (and/or a population of users') historical data to provide more accurate and relevant advice. The system may further identify trends in the user's data and provide recommendations 120 on how to maintain normal metabolic levels. For example, if the system detects a trend of high glucose levels after a particular meal, the system may recommend changes to the user's diet to help maintain normal glucose levels. Further, the machine learning algorithms used by the system are also designed to adapt to changing circumstances. The system may learn from user's feedback and adjust its coaching to provide more accurate and relevant advice. Furthermore, the coaching provided by the system is personalized based on the user's data and may be adjusted based on the user's feedback. In addition, the machine learning algorithms used by the system are designed to provide feedback to user's physicians or doctors. The system shares the user's glucose and metabolic health data with the physician, allowing them to monitor the user's progress and adjust their care plan accordingly. The system also provides feedback to the physician, helping them to understand how the user's actions are affecting their glucose and metabolic health.

Although the description above describes embodiments where the system obtains the real-time glucose levels of the user 200 from the wearable device 108, in certain embodiments, if the user 200 does not possess the wearable device 108, the user 200 may manually provide data related to user's glucose level to the system. The presence of the wearable device 108 is not necessary for the system to operate efficiently, and hence should not be construed as limiting.

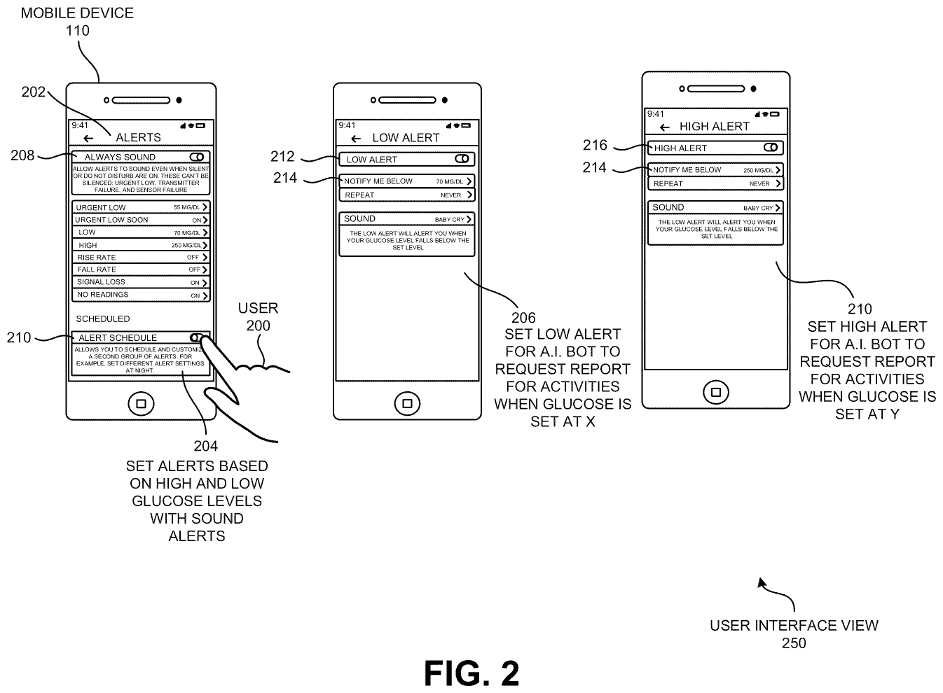

FIG. 2 is a user interface view 250 of the artificial intelligence-powered health system 130 of FIG. 1 illustrating a user 200 setting up an alert 202 using his mobile device 110, according to one embodiment. The wearer 114 may use the mobile application 116 on his mobile device 110 to set alerts 202 on the artificial intelligence-powered health system 130, according to one embodiment. As shown in 204 of FIG. 2, the user 200 may set alerts 202 based on high and low glucose levels along with sound alerts using the “always sound” 208 tab. The “alert schedule” 210 tab may allow the user 200 to schedule and customize a second group of alerts. For example, the user 200 may set different alert settings at night. In 206, the user 200 may set a low alert 212 for the system to request a report for activities when glucose is set below a certain level (e.g., 70 mg/dL) using the “notify me below” 214 tab. The user 200 may set a low numerical (e.g., 70 mg/dL) alert to trigger the system to request input from the user 200. Similar to the view 206, in 210 the user may set a high alert for the AI-powered health system 130 to request a report for activities when glucose is set above a certain level (e.g., 140 mg/dL). Further, the user 200 may set a high numerical (e.g., 250 mg/dL) alert to trigger the system to request input from the user 200, according to one embodiment.

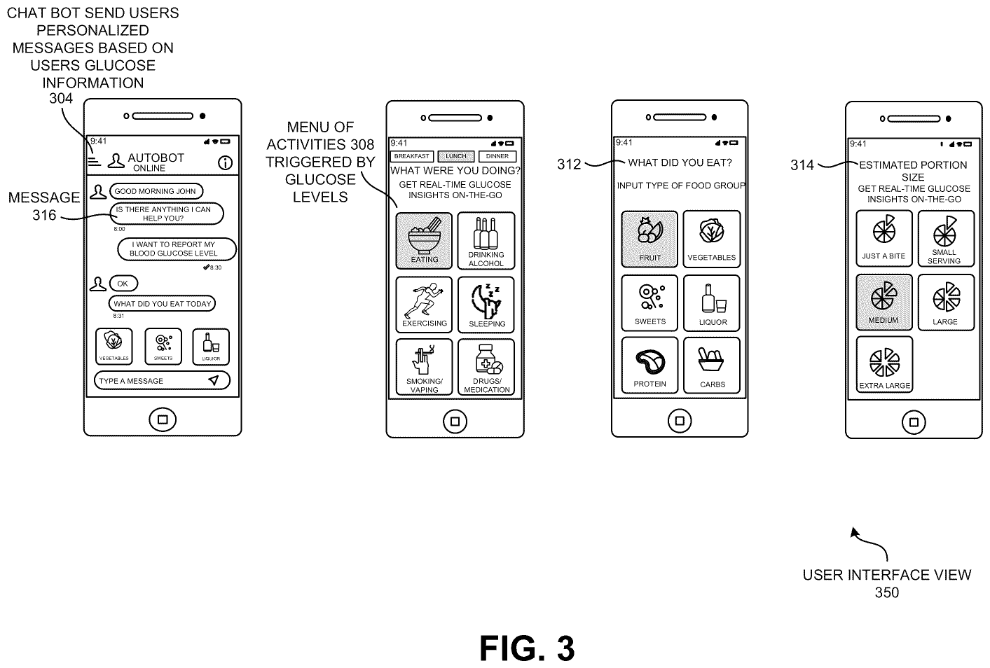

FIG. 3 is another user interface view 350 of the artificial intelligence powered health system 130 of FIG. 1, according to one embodiment. FIG. 3 illustrates the user interface of the mobile device 110 in which the user 200 is able to input details of food consumed by the user 200. The automated chatbot of the artificial intelligence powered health system 130 may send the user a personalized message 316 based on the user's glucose information as shown in 304. The system may check whether the user 200 desires to input details of food consumed/activities performed by the user 200 via photos or manually enter the details. The artificial intelligence powered health system 130 may automatically present the user with a menu of activities 308 triggered by glucose level recorded by its continuous glucose monitoring device 112. The menu of activities 308 may include activities such as eating, drinking alcohol, exercising, sleeping, smoking/vaping, drugs/medication etc. If the user selects eating activity, the user 200 may be directed to specify the food type 312 and its estimated portion size 314 to get real-time glucose level insights, according to one embodiment. In another example embodiment, the user 200 may select a drinking alcohol activity and the user 200 is further prompted to select the type 312 of alcohol and specify an estimated portion size 314 to get real-time glucose level insights.

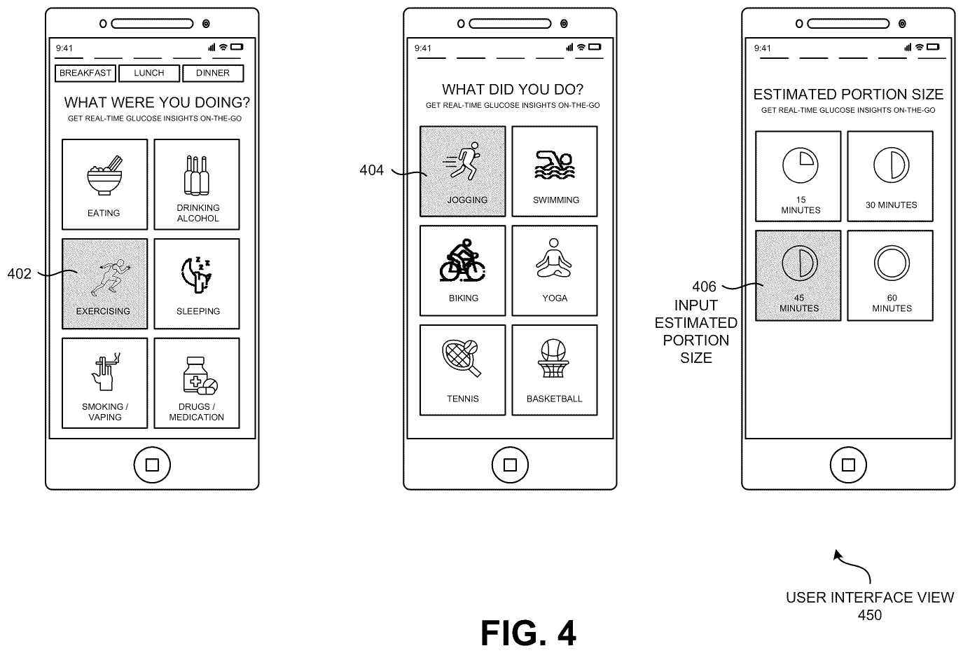

FIG. 4 is another user interface view 450 of the artificial intelligence powered health system 130 of FIG. 1 illustrating the user 200 submitting the details of activity type from the menu of activities 308, according to one embodiment. In an exemplary embodiment, if the user 200 selects the exercising 402 activity in the menu of activities 308, the automated chatbot of the artificial intelligence powered health system 130 may present the user 200 with a number of options of exercise such as jogging, swimming, biking, yoga, tennis, and basketball, etc. The user 200 may select the type 312 of activity and the estimated portion size 314 of the activity he/she is performing to receive a real-time glucose level insights. The user 200 may input jogging 404 activity and an input estimated portion size of 45 minutes 406 from the menu, according to one embodiment.

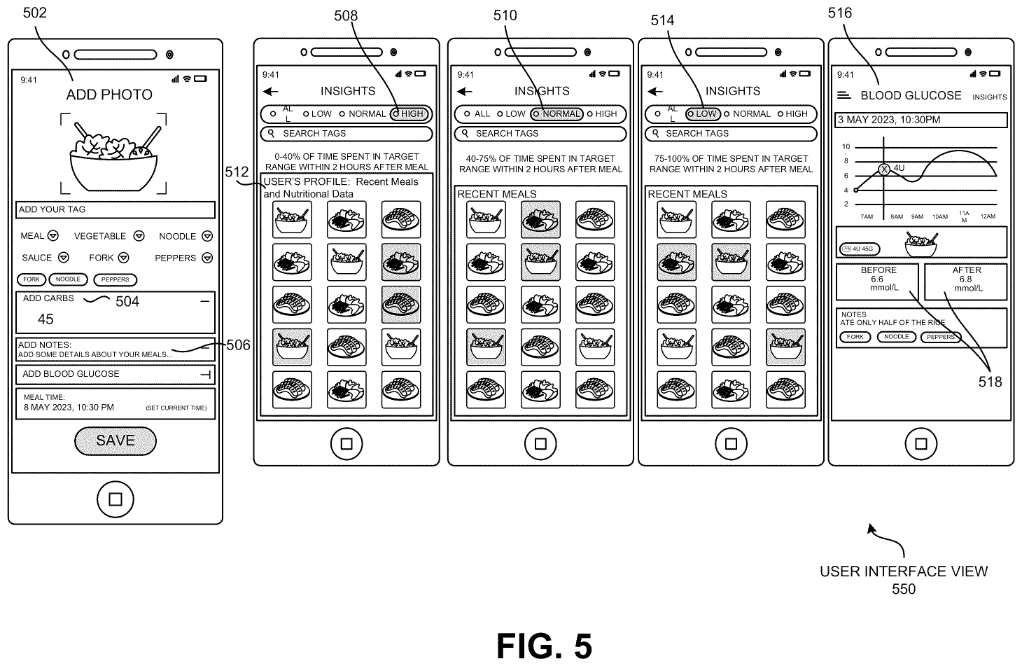

FIG. 5 is another user interface view 550 of the artificial intelligence-powered health system 130 of FIG. 1 illustrating the photographs 502 of food items being uploaded on the system that are consumed by the user 200, according to one embodiment. The user 200 may input the details of the food items consumed by him/her using the camera of his mobile device 110. The AI model 126 of the AI server 100 “reads” the images captured by the camera via computer vision. The AI model 126 of the artificial intelligence-powered health system 130 may extract nutritional information of the food consumed by the user 200 from the image uploaded by the user 200, such as carbs 504. The user 200 may be prompted to add notes about the meals consumed via 506 tab. The artificial intelligence-powered health system 130 may save this nutritional data and corresponding images of the meals to the user's profile 512.

According to one exemplary embodiment, the artificial intelligence-powered health system 130 may display the glucose level details of a plurality of food items as shown on the user interface of the mobile device 110. The system may alert 202 the user 200 which food items are negatively affecting user's glucose levels by displaying the food items saved in the user's profile 512. The 508 tab depicts the artificial intelligence-powered health system 130 displaying photos 502 of food items that are causing high glucose levels over time. The 510 tab depicts the artificial intelligence-powered health system 130 displaying photos 502 of food items that are causing normal glucose levels over time. The 514 tab depicts the artificial intelligence-powered health system 130 displaying photos 502 of food items that are causing low glucose levels over time. The 516 depicts the artificial intelligence-powered health system 130 displaying the glucose levels before and after 518 eating a specific meal, according to one embodiment.

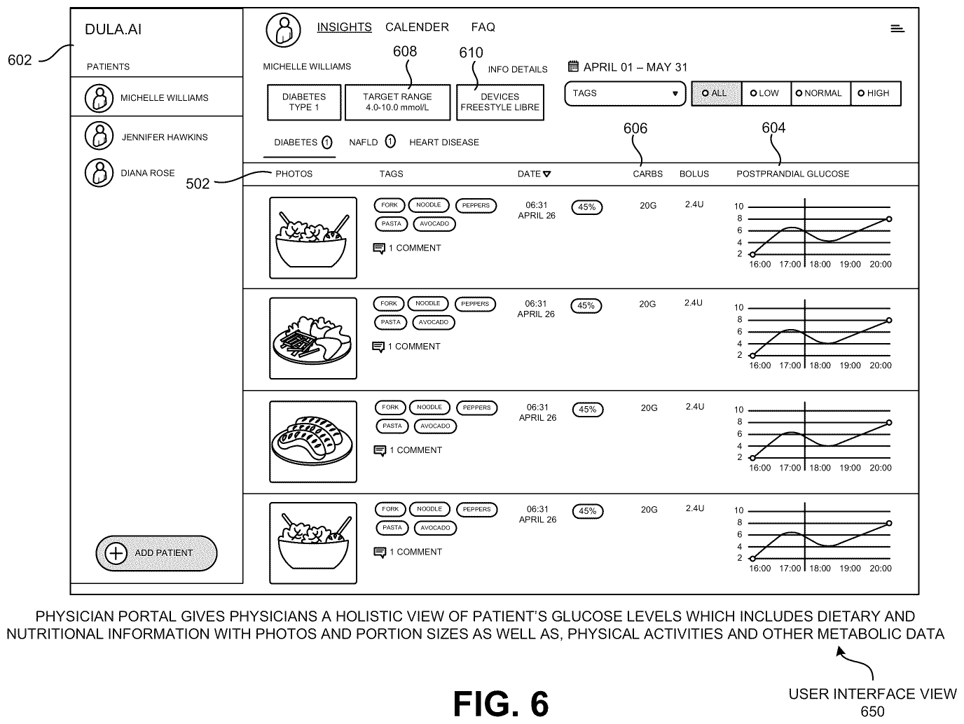

FIG. 6 is a user interface view 650 of a physician portal of the artificial intelligence-powered health system 130 of FIG. 1, according to one embodiment. View 602 depicts a physician portal giving a physician a holistic view of user's glucose levels, which includes dietary and nutritional information 606 with photos 502 and portion sizes, target range 608, postprandial glucose 604 levels, device type 610 as well as physical activities and other metabolic data, according to one embodiment.

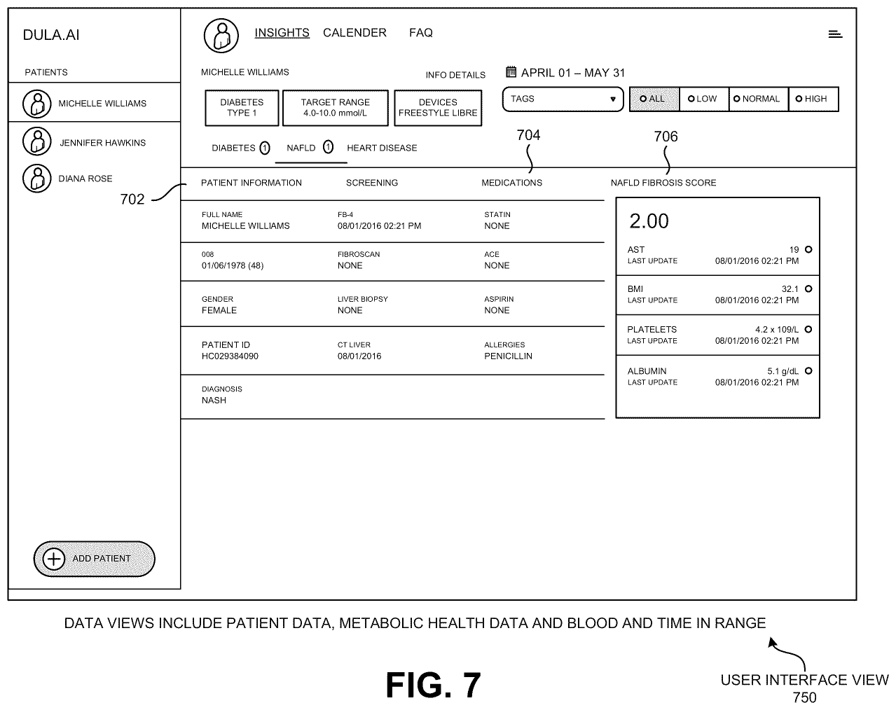

FIG. 7 is another user interface view 750 of the physician portal of the artificial intelligence-powered health system 130 of FIG. 1, according to one embodiment. FIG. 7 illustrates various tabs for the physician (e.g., medical professional 124) to navigate the patients' data in the artificial intelligence-powered health system 130. The physician is able to access patient information 702 including metabolic health data, blood and time in range, medications 704, and clinical data 706 using this interface, according to one embodiment.

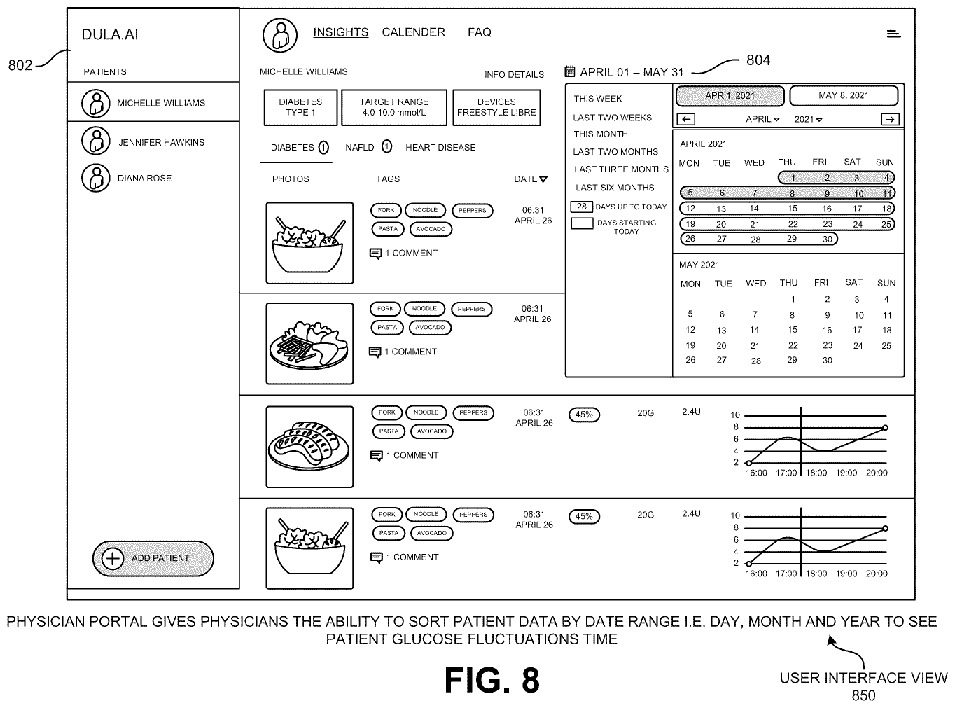

FIG. 8 is a user interface view 850 of a physician portal of the artificial intelligence powered health system 130 of FIG. 1 illustrating the features offered by the system to sort patient data, according to one embodiment. View 802 depicts a physician portal giving the physician the ability to sort patient data by date range, i.e., day, month and year to see patient glucose fluctuations over time. The physician may be able to set a date range using the 804 tab to review the glucose fluctuations for a particular patient over time, according to one embodiment.

In another embodiment, the physician portal gives the physician the ability to search by food, drinks, and activities of a wearer 114 to see how food affects the user's glucose levels.

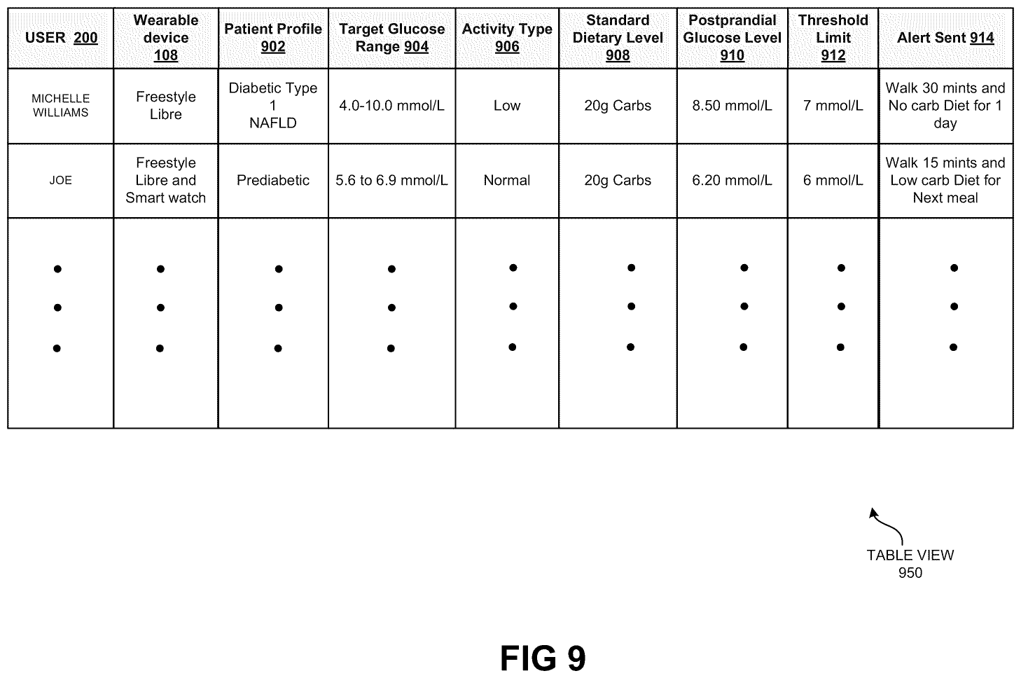

FIG. 9 is a table view 950 of the artificial intelligence-powered health system 130 of FIG. 1, according to one embodiment. A number of fields are illustrated in the table view 950 including fields associated with the user 200 field, a wearable device 108 field, a patient profile 902 field, a target glucose range 904 field, an activity type 906 field, a standard dietary level 908 field, a postprandial glucose level 910 field, a threshold limit 912 field, and a recommendation 120 field according to one embodiment.

Particularly, FIG. 9 illustrates an example of two records—Michelle Williams and Joe. The record of Michelle Williams illustrates that she is using a wearable device 108 Freestyle Libre. Michelle is a diabetic type I and is suffering from NAFLD. Her target glucose range 904 is 4.0-10.0 mmol/L and her activity type 906 is low. Her recommended standard dietary level 908 is 20 g carbs. Post a meal, her wearable device 108 may have detected her postprandial glucose level 910 to be 8.50 mmol/L which is above the threshold limit 912 of 7 mmol/L. Upon detection of a spike in the glucose level, the artificial intelligence-powered health system 130 may automatically trigger an alert 202 to Michelle with a recommendation 120 to walk for approximately 30 minutes and avoid carbs for one day to maintain her glucose level within the threshold limit 912, making her aware of an immediate health risk, according to one embodiment.

The record of Joe illustrates that he is using a wearable device 108 Freestyle Libre communicatively coupled to his smartwatch. Joe is a prediabetic. His target glucose range 904 is 5.6-6.9 mmol/L and his activity type 906 is normal. His recommended standard dietary level 908 is 20 g carbs. Post a meal, his wearable device 108 may have detected his postprandial glucose level 910 to be 6.20 mmol/L which is above the threshold limit 912 of 6 mmol/L. Upon detection of a spike in the glucose level, the artificial intelligence-powered health system 130 may automatically trigger an alert 202 to Joe with a recommendation 120 to walk for approximately 15 minutes and avoid carbs for his next meal to maintain his glucose level within the threshold limit 912, according to one embodiment.

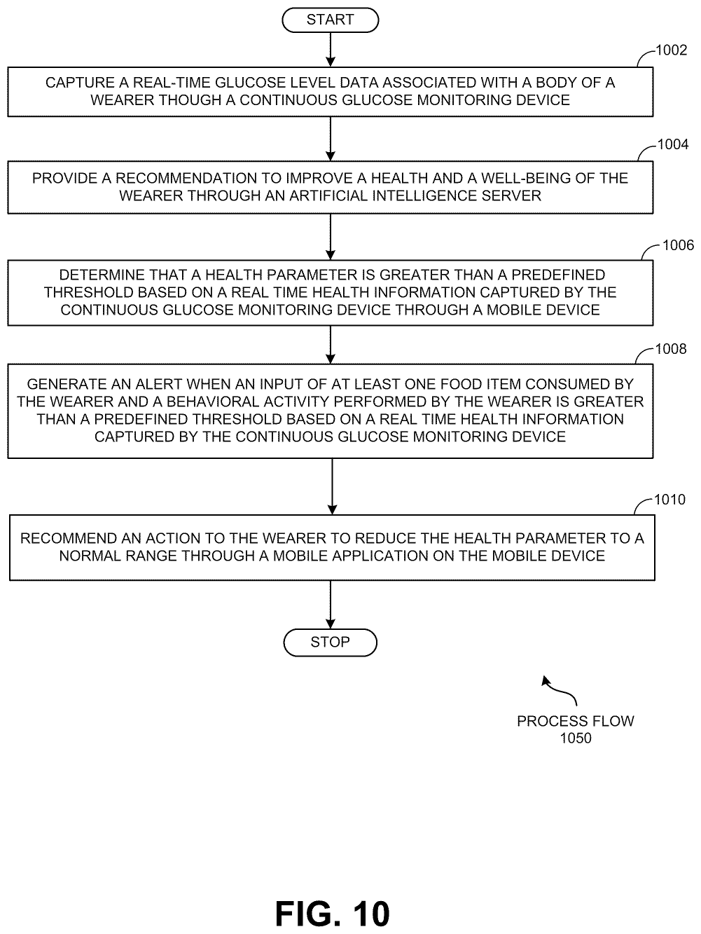

FIG. 10 is a process flow 1050 of the artificial intelligence-powered health system 130 of FIG. 1 illustrating the steps involved in generating a recommendation 120 to a wearer 114 of a continuous glucose monitoring device 114, according to one embodiment. In operation 1002, an artificial intelligence-powered health system 130 may capture real-time glucose level data (e.g., real-time health information 118) associated with a body of a wearer 114 through a continuous glucose monitoring device 112, according to one embodiment.

In operation 1004, the artificial intelligence-powered health system 130 may provide a recommendation 120 to improve a health and a well-being of the wearer 114 through an artificial intelligence server 100. In operation 1006, the artificial intelligence-powered health system 130 may determine that a health parameter is greater than a predefined threshold (e.g., threshold limit 912) based on a real-time health information 118 captured by the continuous glucose monitoring device 112 through a mobile device 110, according to one embodiment.

In operation 1008, the artificial intelligence-powered health system 130 may generate an alert 202 when an input of at least one food item consumed by the wearer 114 and a behavioral activity performed by the wearer 114 is greater than a predefined threshold based on a real-time health information 118 captured by the continuous glucose monitoring device 112, according to one embodiment.

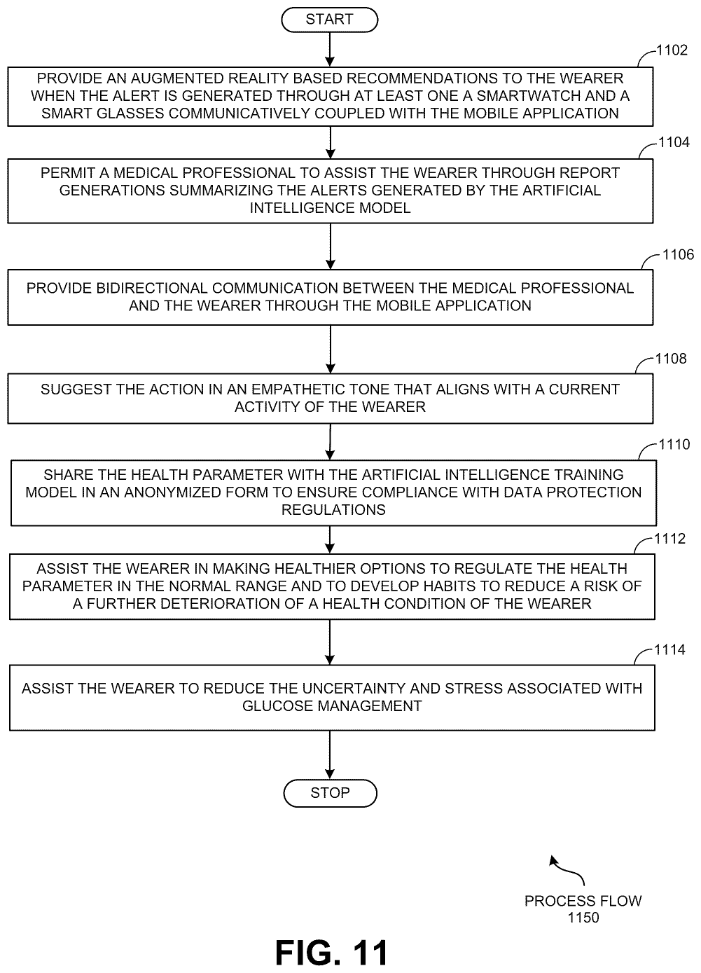

FIG. 11 is another process flow 1150 of the artificial intelligence-powered health system 130 of FIG. 1 illustrating the steps involved in assisting the wearer 114 to reduce the uncertainty and stress associated with glucose management, according to one embodiment.

In operation 1102, the artificial intelligence-powered health system 130 may provide an augmented reality-based recommendation 120 to the wearer 114 when the alert 202 is generated through at least one a smartwatch 109 and a smart glasses 107 communicatively coupled with the mobile application 116, according to one embodiment.

In operation 1104, the artificial intelligence-powered health system 130 may permit a medical professional 124 to assist the wearer 114 through report generations summarizing the alerts 202 generated by the artificial intelligence model 126. In operation 1106, the artificial intelligence-powered health system 130 may provide bidirectional communication between the medical professional 124 and the wearer 114 through the mobile application 116, according to one embodiment.

In operation 1108, the artificial intelligence-powered health system 130 may suggest the action in an empathetic tone that aligns with the current activity of the wearer 114. In operation 1110, the artificial intelligence-powered health system 130 may share the health parameter with the artificial intelligence training model in an anonymized form to ensure compliance with data protection regulations, according to one embodiment.

In operation 1112, the artificial intelligence-powered health system 130 may assist the wearer 114 in making healthier options to regulate the health parameter in the normal range and to develop habits to reduce a risk of a further deterioration of a health condition of the wearer 114. In operation 1114, the artificial intelligence-powered health system 130 may assist the wearer 114 to reduce the uncertainty and stress associated with glucose management, according to one embodiment.

A plain English example will now be described. Joe is a 45-year-old man who has been diagnosed as prediabetic, according to one embodiment. He uses a Continuous Glucose Monitor (CGM) and the Dula™ AI Model 126 integrated into a mobile application to manage his glucose levels, according to one embodiment. Joe is committed to making lifestyle changes to prevent the progression to diabetes and improve his overall health, according to one embodiment. Joe's CGM continuously tracks his glucose levels and sends real-time data to the mobile application on his smartphone, according to one embodiment. One afternoon, Joe's glucose levels start to rise after lunch, according to one embodiment. The data from his CGM is instantly relayed to the cloud-based Dula™ AI Model 126 and/or an edge-based processor directly running on the mobile phone), which processes and analyzes it along with contextual information such as Joe's recent dietary inputs, physical activity, and GPS location, according to one embodiment.

In an edge-based embodiment, the computational processing is shifted closer to the data source rather than relying solely on cloud-based servers, according to one embodiment. This approach reduces latency, enhances privacy, and improves the responsiveness of the system, according to one embodiment. For Joe, a prediabetic, this means faster and more immediate insights and recommendations as his glucose levels are monitored in real-time, according to one embodiment. The Dula™ AI Model 126, having learned from Joe's historical data and patterns, predicts a potential glucose spike based on the current data, according to one embodiment. Before Joe's glucose levels reach a critical point, the mobile application generates a proactive alert, according to one embodiment. Joe receives a push notification on his smartphone: “Alert: Potential Glucose Spike Detected,” according to one embodiment. Upon receiving the alert, Joe opens the app, which engages him interactively, according to one embodiment. The app asks, “Hi Joe, we noticed a potential spike in your glucose levels, according to one embodiment. What did you have for lunch?” Joe responds by inputting that he had a large pasta meal, according to one embodiment. The app then provides personalized recommendations: “Consider taking a 15-minute walk to help manage your glucose levels.” It also suggests a lighter, low-carb snack for his next meal to prevent further spikes, according to one embodiment. As Joe follows the recommendations and takes a walk, his CGM continues to monitor his glucose levels, according to one embodiment. The subsequent data shows a stabilization in his glucose levels, which is sent back to the Dula™ AI Model 126, according to one embodiment. This feedback loop helps the AI model refine its predictions and recommendations for future instances, according to one embodiment.

Joe also wears a smartwatch that tracks his physical activity and heart rate, according to one embodiment. This additional data is integrated into the Dula™ AI Model 126, providing a more comprehensive understanding of Joe's physiological state and enhancing the accuracy of predictions and recommendations, according to one embodiment. While walking, Joe uses his Amazon Alexa-enabled headphones to ask for a summary of his glucose levels for the day, according to one embodiment. The voice-activated assistant provides a brief report, reinforcing Joe's engagement and awareness, according to one embodiment. Joe logs into the app's community platform to share his experience and receive support from other users, according to one embodiment. He joins a challenge to maintain stable glucose levels for a week, motivated by the encouragement and tips from peers, according to one embodiment. Joe earns points for following the app's recommendations and stabilizing his glucose levels, according to one embodiment. These points accumulate and unlock rewards, such as discounts on healthy food options or fitness gear, making the management process more engaging and rewarding, according to one embodiment. The next day, Joe scans a food item using his smartphone's camera, according to one embodiment. The app's AR feature overlays information showing the potential impact of the food on his glucose levels, helping Joe make informed dietary choices, according to one embodiment.

Through the Dula™ AI Model 126, Joe receives real-time insights and proactive alerts about his glucose levels, enabling him to take immediate actions to prevent spikes, according to one embodiment. The continuous monitoring and personalized recommendations help Joe manage his prediabetic condition effectively, according to one embodiment. The integration of creative approaches such as wearable devices, voice-activated assistance, community support, gamification, and AR features enriches Joe's experience and enhances his ability to maintain a healthy lifestyle, according to one embodiment. This proactive, AI-driven approach empowers Joe to make informed decisions and take control of his health, reducing the risk of developing diabetes, according to one embodiment.

Empathetic Embodiment: Inference Phase with the Dula™ AI Model 126

In this embodiment, the Dula™ AI Model 126 not only monitors Joe's glucose levels and provides recommendations but also adopts an empathetic and supportive role, functioning as a life coach, according to one embodiment. This approach focuses on using the right tone and frequency of communication to engage Joe effectively, making the AI interaction feel more personal and motivating, according to one embodiment. Joe's Continuous Glucose Monitor (CGM) tracks his glucose levels and sends this data to the Dula™ AI Model 126 integrated into his mobile application, according to one embodiment. The AI processes this data in real-time, ensuring that Joe receives timely and relevant insights into his health, according to one embodiment. When the Dula™ AI Model 126 detects a potential glucose spike, it sends a proactive alert to Joe with a personalized and empathetic tone, according to one embodiment. Instead of a generic notification, Joe receives a message like, “Hey Joe, it looks like your glucose levels are rising. Don't worry, we've got this! How about we take a short walk together?,” according to one embodiment. The tone of the message is supportive and encouraging, designed to make Joe feel understood and motivated rather than alarmed, according to one embodiment.

The Dula™ AI Model 126 provides Joe with personalized recommendations that are delivered with empathy and understanding, according to one embodiment. For instance, after Joe logs that he had a large pasta meal, the AI might say, “It's great that you enjoyed your meal, Joe! To help balance your glucose levels, let's try a 15-minute walk. You can do it!,” according to one embodiment.

If Joe indicates he is feeling stressed, the AI might respond, “I understand, Joe. Stress can impact your glucose levels. How about some deep breathing exercises to help you relax? I'm here with you every step of the way.”, according to one embodiment.

As Joe interacts with the app and follows its recommendations, the AI continuously learns from these interactions, according to one embodiment. This feedback loop helps the AI refine not only its predictive capabilities but also its communication style, according to one embodiment. The Dula™ AI Model 126 adapts its tone and frequency based on Joe's responses and engagement patterns. If Joe responds positively to frequent check-ins, the AI increases the interaction frequency, according to one embodiment. Conversely, if Joe prefers fewer interactions, the AI adjusts accordingly to avoid being intrusive, according to one embodiment.

The AI tailors its communication style to Joe's preferences, according to one embodiment. For example, if Joe responds better to humor, the AI incorporates light-hearted comments. If Joe prefers a more formal tone, the AI adjusts its language accordingly, according to one embodiment. The AI uses positive reinforcement to motivate Joe. When Joe makes a healthy choice or follows a recommendation, he receives encouraging messages like, “Great job, Joe! You're making fantastic progress. Keep it up!”

The AI helps Joe set realistic and personalized health goals, according to one embodiment. It regularly checks in on his progress, providing support and encouragement, according to one embodiment. For example, “Joe, let's aim for a week of balanced meals. You're doing great, and I'm here to help you every step of the way.”

The AI uses empathetic language to acknowledge Joe's feelings and experiences, according to one embodiment. For instance, if Joe logs feeling tired, the AI might say, “I hear you, Joe. It's okay to feel tired. Let's take it easy today and focus on some gentle activities.”

The AI celebrates Joe's achievements with virtual rewards and acknowledgments, according to one embodiment. “Congratulations, Joe! You've managed your glucose levels well this week. Keep up the fantastic work!” The AI engages Joe in interactive activities like mindfulness exercises, guided meditations, or short educational sessions about nutrition and glucose management, according to one embodiment. “Let's take a few minutes to meditate together, Joe. It will help you feel more relaxed and balanced.” In this empathetic embodiment, the Dula™ AI Model 126 functions as a life coach for Joe, using the right tone and frequency of communication to provide support and motivation. Real-time monitoring and proactive, empathetic alerts ensure that Joe receives timely and relevant insights, according to one embodiment. Personalized behavioral recommendations are delivered with empathy and understanding, helping Joe manage his glucose levels effectively, according to one embodiment. The continuous learning and emotional adaptation capabilities of the AI ensure that Joe's interactions are always supportive and encouraging, according to one embodiment. Creative approaches, such as adaptive communication, positive reinforcement, empathy-driven interactions, personalized goal setting, virtual celebrations, and interactive support, enrich Joe's experience, making the Dula™ AI Model 126 a trusted companion in his health journey, according to one embodiment.

Other features and enhancements make user experience more enjoyable and medically meaningful, according to one embodiment. For example, each increment of glucose measurement may also have a group of corresponding pre-defined, pre-programmed medical responses based on past research results, pre-stored and ready for indexing and access, according to one embodiment. For another example, the artificial intelligence-powered health system 130 can take inputs from speech to text and transcribe the text to be stored in the user's activity logs, according to one embodiment. As yet another example, the A.I. health coach bot can be trained for different types of conversational tones ranging from humorous to sarcastic, with user selectability among them, or even different accents, according to one embodiment. As yet another example, the user 200 may also invite family and friends as followers with the ability to set permissions and thresholds for shared alerts, according to one embodiment.

Smart Glasses and Computer Vision Embodiment: Inference Phase with the Dula™ AI Model 126

In this embodiment, Joe uses smart glasses equipped with computer vision technology, integrated with the Dula™ AI Model 126 to manage his glucose levels more effectively, according to one embodiment. This advanced setup provides Joe with real-time insights and recommendations, leveraging augmented reality (AR) and computer vision to enhance his everyday interactions and decision-making processes, according to one embodiment. Joe's Continuous Glucose Monitor (CGM) tracks his glucose levels and sends the data to his smart glasses and the Dula™ AI Model 126 integrated into the mobile application, according to one embodiment. The smart glasses receive real-time data, enabling immediate processing and feedback, according to one embodiment.

When the Dula™ AI Model 126 detects a potential glucose spike, it sends a proactive alert to Joe's smart glasses. Joe sees a visual notification in his field of view: “Alert: Potential Glucose Spike Detected.” This notification is accompanied by an empathetic message displayed in AR, “Hey Joe, your glucose levels are rising. Let's take a quick walk to help stabilize them.” The visual alert ensures Joe can see the notification immediately, regardless of what he is doing, according to one embodiment.

The Dula™ AI Model 126 provides Joe with personalized recommendations that are visually augmented through his smart glasses 107, according to one embodiment. For example, when Joe is about to have a meal, the computer vision system in the smart glasses 107 can identify the food items on his plate, according to one embodiment. The glasses 107 overlay nutritional information and potential impacts on his glucose levels in real-time: “This pasta may cause a spike in your glucose levels. Consider a smaller portion or adding a side of vegetables,” according to one embodiment

If Joe follows the recommendation and opts for a healthier choice, the glasses 107 provide positive reinforcement: “Great choice, Joe! This will help keep your glucose levels stable.” As Joe interacts with his smart glasses 107 and follows the recommendations, the AI continuously learns from these interactions, according to one embodiment. This feedback loop helps refine both the AI's predictive capabilities and the visual cues provided by the smart glasses 107, according to one embodiment. The AI adapts the content and frequency of the visual alerts based on Joe's engagement and preferences, according to one embodiment. The smart glasses 107 use computer vision to analyze Joe's meals in real-time, according to one embodiment. When Joe looks at his food, the glasses 107 overlay information about each item's nutritional content and its potential impact on his glucose levels, according to one embodiment. This instant feedback helps Joe make informed dietary choices, according to one embodiment.

When Joe's glucose levels start to rise, the smart glasses 107 suggest physical activities in AR. “Joe, how about a 15-minute walk? Follow the highlighted path for a scenic route nearby.” The glasses 107 provide visual cues and directions, making it easy for Joe to follow the recommendations, according to one embodiment. The smart glasses 107 can display interactive educational content about glucose management, tailored to Joe's current situation, according to one embodiment. For example, “Did you know that regular physical activity can help stabilize glucose levels? Here's a quick video on the benefits of walking.”

The smart glasses 107 monitor Joe's environment in real-time, according to one embodiment. If Joe is at a restaurant, the glasses can identify menu items and suggest healthier alternatives, according to one embodiment. “Joe, the grilled chicken is a better option than the fried one to keep your glucose levels in check.” The glasses 107 use facial recognition and biometric data to detect signs of stress, according to one embodiment. If Joe appears stressed, the AI provides calming recommendations, “Joe, you look a bit stressed. Let's take a few deep breaths together,” accompanied by a guided breathing exercise displayed in AR, according to one embodiment. The smart glasses 107 remind Joe to log his meals and activities, according to one embodiment. “Don't forget to log your lunch, Joe. This helps us keep your glucose levels stable.” The reminders are visually unobtrusive and timed to Joe's routines, according to one embodiment.

In this smart glasses 107 and computer vision embodiment, Joe benefits from real-time, visually augmented insights and recommendations provided by the Dula™ AI Model 126, according to one embodiment. The smart glasses 107 equipped with AR and computer vision technology enhance Joe's ability to make informed decisions about his health, according to one embodiment. Proactive and visual alerts, along with personalized behavioral recommendations, help Joe manage his glucose levels more effectively, according to one embodiment. Continuous learning and visual adaptation ensure the system evolves with Joe's needs and preferences, according to one embodiment. Creative approaches, such as AR meal analysis, real-time activity suggestions, interactive educational content, dynamic environment monitoring, stress detection, and personalized reminders, enrich Joe's experience, making the Dula™ AI Model 126 a valuable and interactive tool in his daily health management, according to one embodiment.

Human Coach Embodiment: Inference Phase with the Dula™ AI Model 126

In this embodiment, Joe benefits from a combination of the Dula™ AI Model 126 and the support of a human coach or doctor, according to one embodiment. This integrated approach ensures that Joe receives real-time, AI-driven insights and personalized guidance from a healthcare professional, enhancing his ability to manage his glucose levels and overall health, according to one embodiment. Joe's Continuous Glucose Monitor (CGM) tracks his glucose levels and sends the data to the Dula™ AI Model 126 integrated into his mobile application 116, according to one embodiment. This real-time data is also accessible to Joe's human coach or doctor through a secure platform, according to one embodiment.

When the Dula™ AI Model 126 detects a potential glucose spike, it generates a proactive alert 202. Joe receives a notification on his smartphone, “Alert: Potential Glucose Spike Detected.” Simultaneously, his coach or doctor (e.g., medical professional 124) is notified through their professional dashboard, according to one embodiment. Joe's coach might send a follow-up message through the app: “Hi Joe, I noticed your glucose levels are rising. How are you feeling? Can we discuss what you ate recently and any activities you've done?”

This collaborative approach ensures that Joe receives immediate AI-driven alerts 202 and personal, empathetic follow-up from a healthcare professional, according to one embodiment. The Dula™ AI Model 126 provides Joe with personalized recommendations 120 based on his current data and historical patterns, according to one embodiment. These recommendations 120 are also reviewed by his coach or doctor, who can offer additional context and support, according to one embodiment.

For example, after Joe logs a high-carb meal, the AI suggests, “Consider taking a 15-minute walk to help manage your glucose levels.” His coach might add, “Walking is a great idea, Joe. Let's also discuss your meal choices this week to find some healthier alternatives.”

As Joe interacts with the app and follows recommendations 120, new data is continuously collected, according to one embodiment. This data is analyzed by the AI and shared with Joe's coach or doctor, enabling them to monitor his progress and adjust his care plan as needed, according to one embodiment. During regular check-ins, Joe's coach can provide deeper insights, answer questions, and help interpret the AI's findings. For example, “Joe, I've noticed your glucose levels spike after certain meals. Let's explore some low-carb recipes that you might enjoy,” according to one embodiment

Joe schedules regular virtual consultations with his coach or doctor to review his progress, discuss challenges, and update his care plan, according to one embodiment. The AI provides detailed reports and visualizations of Joe's glucose trends to facilitate these discussions, according to one embodiment. The mobile app 116 includes a secure messaging platform where Joe can communicate with his coach or doctor, according to one embodiment. This platform supports real-time chat, video calls, and document sharing, enabling seamless communication and support, according to one embodiment.

Joe's coach creates custom health plans tailored to his needs, incorporating AI-driven insights and professional expertise, according to one embodiment. These plans include dietary guidelines, exercise routines, and stress management techniques, according to one embodiment. In case of a significant health event, the AI can alert Joe's coach or doctor immediately, ensuring rapid response and intervention, according to one embodiment. “Joe, your glucose levels are critically high. Please follow the emergency steps we've discussed and I'll call you right away.”

Joe's coach provides access to a library of educational resources, including articles, videos, and webinars on diabetes management, according to one embodiment. The AI can recommend specific resources based on Joe's needs and interests, according to one embodiment. The coach offers motivational support, celebrating Joe's achievements and encouraging him during difficult times, according to one embodiment. “Great job keeping your glucose levels stable this week, Joe! Your hard work is really paying off.”

In this embodiment, Joe benefits from the combined power of the Dula™ AI Model 126 and the support of a human coach or doctor, according to one embodiment. Real-time monitoring and AI-driven insights are complemented by personalized guidance and empathetic care from a healthcare professional, according to one embodiment. Proactive alerts 202, collaborative care, personalized recommendations 120, and continuous learning ensure that Joe receives comprehensive support in managing his glucose levels, according to one embodiment. Creative approaches such as regular virtual consultations, an integrated messaging platform, custom health plans, emergency support, educational resources, and motivational support enrich Joe's experience and enhance his ability to achieve his health goals, according to one embodiment. This integrated approach makes the Dula™ AI Model 126 and human coach partnership a valuable and effective tool in Joe's health management, according to one embodiment.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware circuitry (e.g., CMOS based logic circuitry), firmware, software or any combination of hardware, firmware, and software (e.g., embodied in a non-transitory machine-readable medium). For example, the various electrical structures and methods may be embodied using transistors, logic gates, and electrical circuits (e.g., application-specific integrated (ASIC) circuitry and/or Digital Signal Processor (DSP) circuitry).

In addition, it will be appreciated that the various operations, processes and methods disclosed herein may be embodied in a non-transitory machine-readable medium and/or a machine-accessible medium compatible with a data processing system (e.g., data processing device 100). Accordingly, the specification and drawings are to be regarded in an illustrative rather than a restrictive sense.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from; the described systems. Accordingly, other embodiments are within the scope of the following claims.

It may be appreciated that the various systems, methods, and apparatus disclosed herein may be embodied in a machine-readable medium and/or a machine-accessible medium compatible with a data processing system (e.g., a computer system), and/or may be performed in any order.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1. An artificial intelligence powered health system, comprising:
a continuous glucose monitoring device to capture a real-time glucose level data associated with a body of a wearer;
an artificial intelligence server to provide a recommendation to improve a health and a well-being of the wearer;
a mobile device communicatively coupled with the continuous glucose monitoring device and the artificial intelligence server to generate an alert when an input of at least one food item consumed by the wearer and a behavioral activity performed by the wearer exhibits a health parameter which is greater than a predefined threshold based on a real time health information captured by the continuous glucose monitoring device; and
a mobile application on the mobile device to recommend an action to the wearer to reduce the health parameter to a normal range.

2. The artificial intelligence powered health system of claim 1 further comprising:
a smart glasses communicatively coupled with the mobile application to provide augmented reality based recommendations to the wearer when the alert is generated.

3. The artificial intelligence powered health system of claim 1 further comprising:
a community assistance server communicatively coupled the artificial intelligence server to permit a medical professional to assist the wearer through report generations summarizing the alerts generated by the artificial intelligence model, and to provide bidirectional communication between the medical professional and the wearer through the mobile application.

4. The artificial intelligence powered health system of claim 1 wherein the action is provided in an empathetic tone that aligns with a current activity of the wearer.

5. The artificial intelligence powered health system of claim 1 wherein the health parameter is shared with an artificial intelligence training model in an anonymized form to ensure compliance with data protection regulations.

6. The artificial intelligence powered health system of claim 1 wherein the alert to assist the wearer in making healthier options to regulate the health parameter in the normal range and to develop habits to reduce a risk of a further deterioration of a health condition of the wearer.

7. The artificial intelligence powered health system of claim 1 wherein the alert to assist the wearer to reduce the uncertainty and stress associated with glucose management.

8. A glucose monitoring system, comprising:
a continuous glucose monitoring device to capture a real-time glucose level data associated with a body of a wearer;
a mobile device communicatively coupled with the continuous glucose monitoring device to generate an alert when the continuous glucose monitoring device to detect a spike in a glucose level on the body of the wearer when the wearer is consuming a food;
a mobile application on the mobile device to automatically share a contextual data comprising at least one of a location data, a weight data, a visual data, an audio data, a transcript, a voice-response data, and an olfactory data that aligns contemporaneously with a timestamp of the alert and a glucose spike level associated with the alert with an artificial intelligence training model; and
an artificial intelligence server to host the artificial intelligence training model to aggregate the contextual data with other contextual data of the wearer and that of other wearers to form a basis for early predictive warnings of imminent glucose spikes based on a continual tagging and training of a predictive artificial intelligence model formed from tagged versions of the contextual data with other contextual data of the wearer and that of other wearers.

9. The glucose monitoring system of claim 8 further comprising:
a smart glasses communicatively coupled with the mobile application to provide augmented reality based recommendations to the wearer when the alert is generated.

10. The glucose monitoring system of claim 8 further comprising:
a community assistance server communicatively coupled the artificial intelligence server to permit a medical professional to assist the wearer through report generations summarizing the alerts generated by the artificial intelligence model, and to provide bidirectional communication between the medical professional and the wearer through the mobile application.

11. The glucose monitoring system of claim 8 wherein the action is provided in an empathetic tone that aligns with a current activity of the wearer.

12. The glucose monitoring system of claim 8 wherein the health parameter is shared with the artificial intelligence training model in an anonymized form to ensure compliance with data protection regulations.

13. The glucose monitoring system of claim 8 wherein the alert to assist the wearer in making healthier options to regulate the health parameter in the normal range and to develop habits to reduce a risk of a further deterioration of a health condition of the wearer.

14. The glucose monitoring system of claim 8 wherein the alert to assist the wearer to reduce the uncertainty and stress associated with glucose management.

15. A method of artificial intelligence powered health system, comprising:
capturing a real-time glucose level data associated with a body of a wearer though a continuous glucose monitoring device;
providing a recommendation to improve a health and a well-being of the wearer through an artificial intelligence server;
determining that a health parameter is greater than a predefined threshold based on a real time health information captured by the continuous glucose monitoring device through a mobile device;
generating an alert when an input of at least one food item consumed by the wearer and a behavioral activity performed by the wearer is greater than a predefined threshold based on a real time health information captured by the continuous glucose monitoring device; and
recommending an action to the wearer to reduce the health parameter to a normal range through a mobile application on the mobile device.

16. The method of claim 15 further comprising:
providing an augmented reality based recommendations to the wearer when the alert is generated through at least one a smartwatch and a smart glasses communicatively coupled with the mobile application.

17. The method of claim 15 further comprising:
permitting a medical professional to assist the wearer through report generations summarizing the alerts generated by the artificial intelligence model; and
providing bidirectional communication between the medical professional and the wearer through the mobile application.

18. The method of claim 15 further comprising:
suggesting the action in an empathetic tone that aligns with a current activity of the wearer.

19. The method of claim 15 further comprising:
sharing the health parameter with the artificial intelligence training model in an anonymized form to ensure compliance with data protection regulations.

20. The method of claim 15 further comprising:
assisting the wearer in making healthier options to regulate the health parameter in the normal range and to develop habits to reduce a risk of a further deterioration of a health condition of the wearer; and
assisting the wearer to reduce the uncertainty and stress associated with glucose management.
